(19) United States
(12) Patent Application Publication
(10) Pub. No.: US 2016/0354061 A1
(43) Pub. Date: Dec. 8, 2016

(54) Method And Apparatus For Ultrasonic Analysis Of Brain Activity In Stroke Patients

(71) Applicant: George Mason University, Fairfax, VA (US)

(72) Inventors: Joseph Hecker, Fairfax, VA (US); Siddhartha Sikdar, Washington, DC (US)

(21) Appl. No.: 15/172,883

(22) Filed: Jun. 3, 2016

Related U.S. Application Data
(60) Provisional application No. 62/170,153, filed on Jun. 3, 2015.

(51) Int. Cl. A61B 8/08; A61B 8/00; A61B 8/02

(52) U.S. Cl. CPC A61B 8/488; A61B 8/02; A61B 8/565; A61B 8/5207; A61B 8/0808

(57) ABSTRACT

Methods are disclosed comprising transmitting ultrasound waves to a plurality of regions of a brain of a subject via one or more probes, receiving ultrasound echoes corresponding to the transmitted ultrasound waves, determining a parameter based on the ultrasound echoes for each region of the plurality of regions, determining a time course for each parameter, and one or more of: comparing the time courses for each region of the plurality of regions to determine a pulsatility measurement for each region of the plurality of regions and comparing the time courses to one or more of, a known time course in normal brain tissue and a known time course in abnormal brain tissue to classify each region of the plurality of regions as comprising normal brain tissue or abnormal brain tissue.

### CROSS REFERENCE TO RELATED PATENT APPLICATION

This application claims priority to U.S. Provisional Application No. 62/170,153 filed Jun. 3, 2015, herein incorporated by reference in its entirety.

### BACKGROUND

Approximately three quarters of a million people suffer a stroke even year. Stroke has a mortality rate greater than 15% and contributes to significant disabilities in those who survive. Early diagnosis and treatment is critical to improve prognosis, since brain tissue is lost if treatment is not performed promptly. A critical decision that must be made before administering treatment is the differentiation between ischemic stroke associated with a blockage of a blood vessel in the brain and hemorrhagic stroke caused by bleeding in the brain. If the stroke is caused by a blockage due to a blood clot, an anticoagulant, such as tPA, should be administered as soon as possible to dissolve the blood clot. If the stroke is hemorrhagic, anticoagulant therapy could be fatal and should not be administered. Currently this differentiation between ischemic and hemorrhagic stroke only can be performed in a hospital setting using advanced imaging.

In standard practice, stroke diagnosis requires the use of computed tomography (CT) scans. The supporting machinery is large and not conducive to point of care measurements. These scans are consequently performed once a patient has arrived at a hospital. The ability to differentiate between the type of stroke in the shortest amount of time can result in saving as much brain tissue from disease as possible. In addition, once a patient is receiving treatment for stroke, the ability to monitor the brain tissue at the bedside without performing repeated CT scans would lead to better ability to manage the treatment of the patient.

It would be desirable, therefore, to develop new technologies for assessing stroke that overcomes these and other limitations of the prior art.

### SUMMARY

It is to be understood that both the following general description and the following detailed description are exemplary and explanatory only and are not restrictive. Methods, systems, and apparatuses are disclosed comprising transmitting ultrasound waves to a plurality of regions of a brain of a subject via one or more probes, receiving ultrasound echoes corresponding to the transmitted ultrasound waves, determining a parameter based on the ultrasound echoes for each region of the plurality of regions, determining a time course for each parameter, and one or more of: comparing the time courses for each region of the plurality of regions to determine a pulsatility measurement for each region of the plurality of regions and comparing the time courses to one or more of, a known time course in normal brain tissue and a known time course in abnormal brain tissue to classify each region of the plurality of regions as comprising normal brain tissue or abnormal brain tissue.

Additional advantages will be set forth in part in the description which follows or may be learned by practice. The advantages will be realized and attained by means of the elements and combinations particularly pointed out in the appended claims.

### BRIEF DESCRIPTION OF THE DRAWINGS

The accompanying drawings, which are incorporated in and constitute a part of this specification, illustrate embodiments and together with the description, serve to explain the principles of the methods and systems:

FIG. 1 is an example ultrasound apparatus;

FIG. 2 is an example probe configuration for the ultrasound apparatus;

FIG. 3A illustrates an example of the variation of the spectral parameter mid-band-fit during the cardiac cycle in a region of normal brain tissue;

FIG. 3B illustrates an example of the variation of the spectral parameter spectral slope during the cardiac cycle in a region of normal brain tissue;

FIG. 3C is illustrates an example of the variation of the spectral parameter zero-frequency-intercept during the cardiac cycle in a region of normal brain tissue;

FIG. 3D illustrates a time course of estimated brain tissue velocity for three cardiac cycles using the disclosed phase shift method (least squares fit of filter bank outputs, LSFB) compared to method described in the prior art (two-dimensional autocorrelation 2DAC);

FIG. 4A illustrates a 3D geometric view of constrained filtered signals from a pair of consecutive reflected ultrasound echoes (RF frequency from pulse 1 to pulse 2);

FIG. 4B illustrates a 3D geometric view of constrained filtered signals from a pair of consecutive reflected ultrasound echoes (RF frequency of pulse 1 to Doppler frequency);

FIG. 4C illustrates a 3D geometric view of constrained filtered signals from a pair of consecutive reflected ultrasound echoes (RF frequency of pulse 2 to Doppler frequency);

FIG. 5 is an example spatial map;

FIG. 6 is a flowchart illustrating an example method;

FIG. 7 is a flowchart illustrating an example method;

FIG. 8 is a flowchart illustrating an example method;

FIG. 9 is a flowchart illustrating an example method;

FIG. 10 is a flowchart illustrating an example method;

FIG. 11 is a flowchart illustrating an example method; and

FIG. 12 is an example operating environment.

### DETAILED DESCRIPTION

Before the present methods and systems are disclosed and described, it is to be understood that the methods and systems are not limited to specific methods, specific components, or to particular implementations. It is also to be understood that the terminology used herein is for the purpose of describing particular embodiments only and is not intended to be limiting.

As used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

The present methods and systems may be understood more readily by reference to the following detailed description of preferred embodiments and the examples included therein and to the Figures and their previous and following description.

As will be appreciated by one skilled in the art, the methods and systems may take the form of an entirely hardware embodiment, an entirely software embodiment, or an embodiment combining software and hardware aspects. Furthermore, the methods and systems may take the form of a computer program product on a computer-readable storage medium having computer-readable program, instructions (e.g., computer software) embodied in the storage medium. More particularly, the present methods and systems may take the form of web-implemented computer software. Any suitable computer-readable storage medium may be utilized including hard disks, CD-ROMs, optical storage devices, or magnetic storage devices.

Embodiments of the methods and systems are described below with reference to block diagrams and flowchart illustrations of methods, systems, apparatuses and computer program products. It will be understood that each block of the block diagrams and flowchart illustrations, and combinations of blocks in the block diagrams and flowchart illustrations, respectively, can be implemented by-computer program instructions. These computer program instructions may be loaded onto a general purpose computer, special purpose computer, or other programmable data processing apparatus to produce a machine, such that the instructions which execute on the computer or other programmable data processing apparatus create a means for implementing the functions specified in the flowchart block or blocks.

These computer program instructions may also be stored in a computer-readable memory that can direct a computer or other programmable data processing apparatus to function in a particular manner, such that the instructions stored in the computer-readable memory produce an article of manufacture including computer-readable instructions for implementing the function specified in the flowchart block or blocks. The computer program instructions may also be loaded onto a computer or other programmable data processing apparatus to cause a series of operational steps to be performed on the computer or other programmable apparatus to produce a computer-implemented process such that the instructions that execute on the computer or other programmable apparatus provide steps for implementing the functions specified in the flowchart block or blocks.

Accordingly, blocks of the block diagrams and flowchart illustrations support combinations of means for performing the specified functions, combinations of steps for performing the specified functions and program instruction means for performing the specified functions. It will also be understood that each block of the block diagrams and flowchart illustrations, and combinations of blocks in the block diagrams and flowchart illustrations, can be implemented by special purpose hardware-based computer systems that perform the specified functions or steps, or combinations of special purpose hardware and computer instructions.

Throughout the specification, an “ultrasound image” can refer to an image of an object, which is obtained using ultrasound waves. Furthermore, an “object” may be a human, an animal, or a part of a human or animal. For example, the object may be an organ (e.g., the liver, the heart, the brain, the abdomen, and the like), a blood vessel, or a combination thereof. Also, the object may be a phantom. The phantom means a material having a density, an effective atomic number, and a volume that are approximately the same as those of an organism.

The present disclosure relates to methods, systems, and apparatuses for using ultrasound to assess brain activity to differentiate between ischemic and hemorrhagic strokes. The methods, systems, and apparatuses can use novel ultrasound methods and apparatuses to derive brain tissue properties with the application of detecting, localizing, and characterizing affected tissue. Normal brain tissue has a characteristic pulsation due to the cardiac cycle. The cardiac cycle defines the blood dynamics associated with each beat of the heart. In a simple description, the cardiac cycle is comprised of two phases: systole and diastole. Systole describes period over which the heart contracts and results in increased blood pressure. Correspondingly, systole results in increased blood flow through vessels and tissue. Diastole describes the period when the heart muscles relax and the pressure begins to decrease. A single cardiac cycle refers to the period from the onset of systole from one heart beat to the onset of systole in the next heartbeat. Both blood vessels and brain tissue will exhibit pulsations that reflect the changes in the volume of blood that is passing through them at a given moment in time. If blood flow is disrupted to a local region of tissue, the nature of the pulsation of that local region of tissue is expected to change significantly. The methods, systems, and apparatuses include novel signal processing methods to robustly quantify these pulsations. This characterization is significantly different from traditional ultrasound imaging techniques, which do not provide adequate performance. In an aspect, methods are disclosed that can examine brain tissue characteristics using ultrasonic signals. The methods are based on changes in brain tissue characteristics during the cardiac cycle and their measurement using backscattered ultrasonic echoes that enables measurement of tissue property changes in conditions such as stroke.

A patient suffering an ischemic stroke has a blood clot preventing blood to flow to brain tissue while a hemorrhagic stroke involves bleeding in the brain. For ischemic stroke, the region of the brain that is impacted is expected to exhibit decreased pulsatile tissue motion during the cardiac cycle. The methods disclosed can use ultrasound signals and image processing to detect the velocity of brain tissue motion during these pulsations and/or changes in spectral parameters that relate to impedance, density of scatterers, and/or size of scatterers in the brain. Using these methods, it has been shown that normal brain tissue exhibits a pattern of cyclic parameter changes during the cardiac cycle. Brain tissue that has been affected by-stroke is not expected to have similar cyclic parametric changes as a result of restricted blood flow and thus, can be distinguished from normal brain tissue. For hemorrhagic stroke, blood enters the brain and compresses the tissue it surrounds. Such behavior will modify the pattern of cyclic parameter changes during the cardiac cycle, and this modified pattern is expected to be different than normal brain tissue. Therefore incorporation of spectral parameters into assessment of tissue properties can lead to the differentiation between the two types of strokes.

The methods, systems, and apparatuses disclosed can perform stroke type detection and stroke localization. In an aspect, the methods can comprise calculating tissue parameters, normalization of the tissue parameters relative to a measured reference, and evaluation of the tissue parameters over time. Comparison of tissue through the brain can be made such that areas that behave abnormally can be identified. The manner in which the measures deviate between affected tissue and normal tissue provides can be used to differentiate between stroke type. In an aspect, the measures can be calculated for each pulse individually. This can reduce errors associated with motion and allow for slower frame rates of acquisition.

In an aspect, the methods, systems, and apparatuses can utilize Transcranial pulsatility imaging (TPI). TPI directly measures tissue velocity by means of Doppler frequency estimation. This measure relies explicitly on the phase relationship between successive ultrasonic pulses. However, the methods disclosed are not explicitly derivative of the Doppler information (e.g., tissue velocity). Ultrasonic spectral parameter estimation (USPE) can be used to assess tissue properties such as impedance, scatterer density, and size of scatterer. Existing methods focus on tissue spectral with the underlying assumption that the parameters are stationary over time. The methods, systems, and apparatuses disclosed can use the assumption that these parameters are in fact dynamic. The cyclic variation of integrated backscatter (CVIB) is a closely related topic to the present disclosure as CVIB measures tissue properties across multiple pulses but not in a Doppler sense. The methods, systems, and apparatuses can be based on cyclic variation in brain tissue properties. The methods, systems, and apparatuses further include developing images based on cyclic variations. Both TPI and USPE are imaging based approaches but do not focus on cyclic variations of tissue parameters. CVIB provides information on cyclic variations, but does not focus on generating images. The present methods, systems, and apparatuses are more robust than TPI measures that are reliant on information derivative of individual pulses. Motion between ultrasound pulses can significantly degrade the velocity estimate whereas spectral parameters are calculated at significantly higher rate.

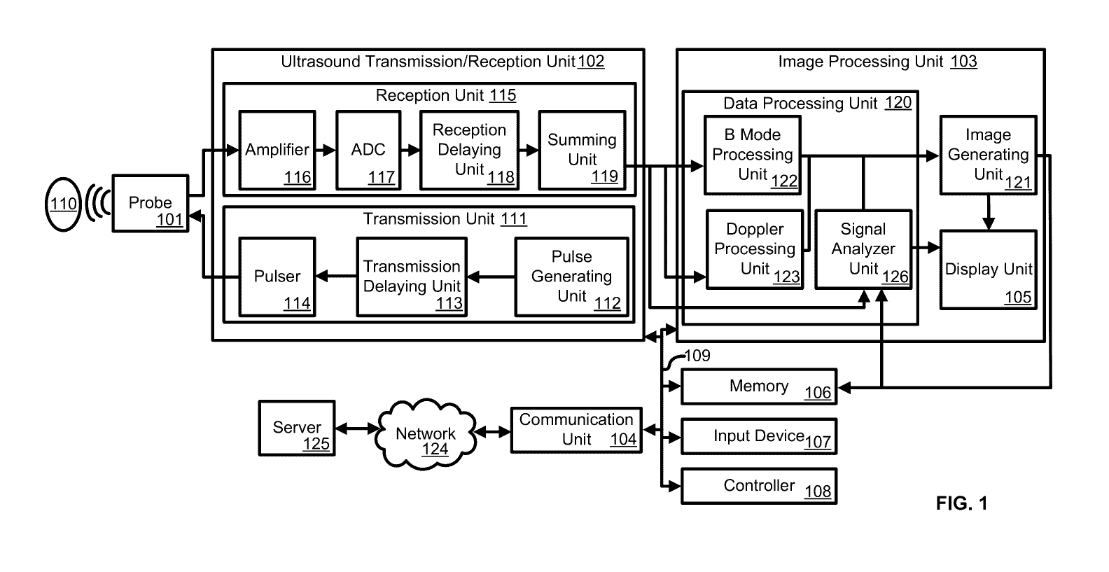

In an aspect, the methods and systems can comprise an ultrasound apparatus. FIG. 1 illustrates an example ultrasound apparatus 100. The ultrasound apparatus 100 may further include configurations not shown in FIG. 1 or may omit some of the configurations illustrated in FIG. 1. Also, the configurations illustrated in FIG. 1 may be substituted by equivalents.

The ultrasound apparatus 100 may include one or more probes 101, an ultrasound transmission/reception unit (e.g., transceiver) 102, an image processing unit 103, a communication unit 104, a display unit 105, a memory 106, an input device 107, and a controller 108, which may be connected to one another via buses 109.

The ultrasound apparatus 100 may be a cart type apparatus or a portable type apparatus. Examples of portable ultrasound apparatuses may include, but are not limited to, a picture archiving and communication system (PACS) viewer, a smartphone, a laptop computer, a personal digital assistant (PDA), and a tablet PC.

The probe 101 transmits ultrasound waves to an object 110 in response to a driving signal applied by the ultrasound transmission/reception unit 102 and receives echo signals reflected by the object 110. The probe 101 includes a plurality of transducers, and the plurality of transducers oscillate in response to electric signals and generate acoustic energy, that is, ultrasound waves. Furthermore, the probe 101 may be connected to the main body of the ultrasound apparatus 100 by wire or wirelessly, and according to embodiments, the ultrasound apparatus 100 may include a plurality of probes 101.

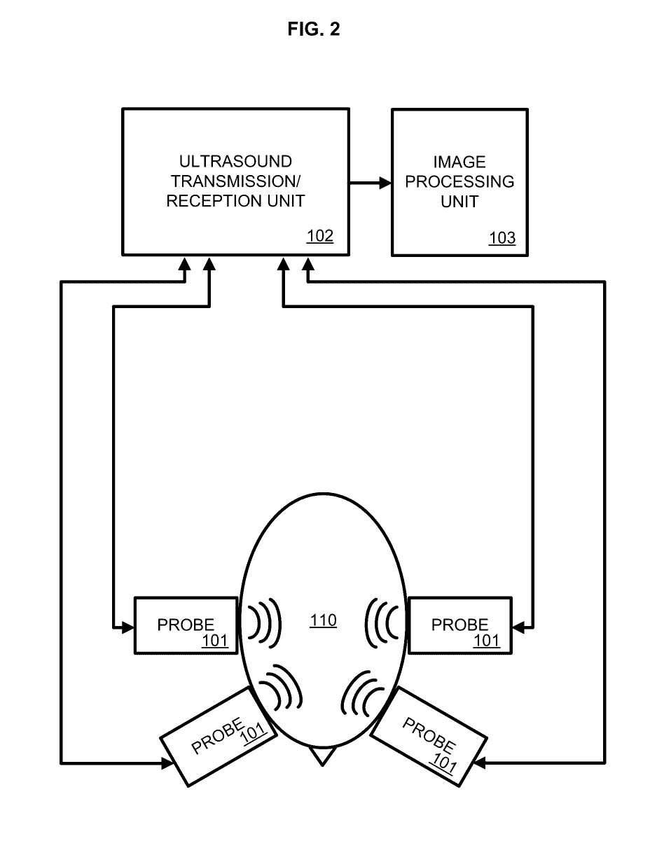

FIG. 2 illustrates an example configuration for one or more probes 101. Hie object 110 can comprise a head (e.g., a human head). And the one or more probes 101 can be positioned at either side of the object 110 and at either side of a forehead of the object 110. The one or more probes can be coupled to the ultrasound transmission/reception unit 102 and can operate as disclosed herein, in an aspect, the one or more probes 101 can be configured as a helmet or belt that can be affixed to the object 110 (e.g., a subject's head). In an aspect, the one or more probes 101 can be integrated into a cylindrical device within which a subject's head is placed, similar in appearance to a magnetic resonance imaging (MRI) head coil.

Returning to FIG. 1, a transmission unit 111 supplies a driving signal to the probe 101. The transmission unit 111 includes a pulse generating unit 112, a transmission delaying unit 113, and a pulser 114. The pulse generating unit 112 generates pulses for forming transmission ultrasound waves based on a predetermined pulse repetition frequency (PRF), and the transmission delaying unit 113 delays the pulses by delay times necessary for determining transmission directionality. The pulses which have been delayed correspond to a plurality of piezoelectric vibrators included in the probe 101, respectively. The pulser 114 applies a driving signal (or a driving pulse) to the probe 101 based on timing corresponding to each of the pulses which have been delayed.

A reception unit 115 generates ultrasound data by processing echo signals received from the probe 101. The reception unit 115 may include an amplifier 116, an analog-to-digital converter (ADC) 117, a reception delaying unit 118, and a summing unit 119. The amplifier 116 amplifies echo signals in each channel, and the ADC 117 performs analog-to-digital conversion with respect to the amplified echo signals. The reception delaying unit 118 delays digital echo signals output by the ADC 117 by delay times necessary for determining reception directionality, and the summing unit 119 generates ultrasound data by summing the echo signals processed by the reception delaying unit 118. In some aspects, the reception unit 115 may not include the amplifier 116. In other words, if the sensitivity of the probe 101 or the capability of the ADC 117 to process bits is enhanced, the amplifier 116 may be omitted.

The image processing unit 103 generates one or more ultrasound images by processing ultrasound data generated by the ultrasound transmission/reception unit 102. The image processing unit 103 can comprise a data processing unit 120 and an image generating unit 121. The image processing unit 103 can process the ultrasound data via scan-converting, for example. However, according to aspects, the scan-converting may be omitted. The ultrasound image may be not only a grayscale ultrasound image obtained by scanning an object in an amplitude (A) mode, a brightness (B) mode, and a motion (M) mode, but also a Doppler image showing a movement of an object via a Doppler effect. The Doppler image may be a blood flow Doppler image showing flow of blood (also referred to as a color Doppler image), a tissue Doppler image showing a movement of tissue, or a spectral Doppler image showing a moving speed of an object as a waveform.

The data processing unit 120 can comprise a B mode processing unit 122 and/or a Doppler processing unit 123. The B mode processing unit 122 extracts B mode components from ultrasound data and processes the B mode components. The image generating unit 121 may generate an ultrasound image indicating signal intensities as brightness based on the extracted B mode components. The Doppler processing unit 123 may extract Doppler components from the ultrasound data. The image generating unit 121 may generate a Doppler image indicating a movement of an object as colors or waveforms based on the extracted Doppler components.

The data processing unit 120 can further comprise a signal analyzer unit 126. The signal analyzer unit 126 can receive ultrasound data from the reception unit 115. The signal analyzer unit 126 can analyze the ultrasound data to compare ultrasound reflections from different brain regions to determine whether the brain regions are normal or are affected by ischemic or hemorrhagic stroke.

In an aspect, the signal analyzer unit 126 can estimate an instantaneous intensity of backscattered ultrasound echoes from, a region of brain tissue. The signal analyzer unit 126 conditions the analog backscattered signal for digitization by the ADC 117 through the operations of amplification and of filtering. The received digital signal is converted to analytic form (e.g., complex valued) through a Hilbert transform or quadrature filtering operation. The magnitude of the complex valued received signal is defined as the instantaneous backscattered intensity and may be summed over spatially adjoining samples in depth and/or lateral directions to comprise one image voxel. The instantaneous backscattered intensity is calculated for every voxel in the field of regard of the ultrasound transmission. The time course of the instantaneous backscattered intensity in each voxel will exhibit cyclic oscillations with a period equal to that of the cardiac cycle. The time courses for instantaneous backscattered intensity measures from multiple cardiac cycles may be time synchronized and averaged together. The time synchronization can be achieved through time course resampling or phase adjustments using the peak systolic time instant as a reference. The electrocardiogram signal can be used to obtain a reference indicator of the phases of the cardiac cycle. The duration of the cardiac cycle can also be derived from the intrinsic period of the signals derived from brain tissue motion. The time course, and intrinsic features thereof, of the backscattered intensity across the cardiac cycle are compared for each voxel against: 1) previously stored known variations in normal brain tissue, 2) previously stored patterns or time courses of abnormal brain tissue, 3) previously stored historical patterns or time courses from the same subject, if available, for monitoring treatment. Any number of classification algorithms (including but not limited to Bayesian, neural network, support vector machines, k-nearest neighbor, and binary decision) can be used to determine whether the observed brain tissue region exhibits (a) normal pulsation, (b) abnormal pulsation characteristic of brain tissue affected by ischemic stroke, (c) abnormal pulsation characteristic of brain tissue affected by hemorrhagic stroke or (d) unknown abnormal pulsation.

In another aspect, the signal analyzer unit 126 can estimate the instantaneous frequency spectrum of the backscattered ultrasound echoes from a region of brain tissue, and calculate a number of instantaneous spectral parameters from the reflected ultrasound signal. The signal analyzer unit 126 conditions the analog backscattered signal for digitization by the ADC 117 through the operations of amplification and of filtering. The received digital signal is converted to analytic form (e.g. complex valued) through a Hilbert transform or quadrature filtering operation. The frequency spectrum is calculated for a set of L samples by one or more of a number of methods including but not limited to Fast Fourier Transform, Welch periodogram averaging, or autoregressive spectrum estimation. Features of the resultant spectra are then calculated. Features include, but are not limited to, mid-band fit, spectral slope, and zero-frequency intercept. Mid-band fit, β, is calculated as the average of the N frequency bins of the calculated spectrum, Z. Mid-band fit is measured as

$$β = \frac{1}{N} {\sum }_{n}^{ } {Z}_{n}$$

Spectral slope is calculated as

$$m = {\sum }_{n}^{ } \frac{12 ( n - N / 2 ) {Z}_{n}}{( {N}^{3} + 2 N ) Δ f} ,$$

where Δf is the frequency spacing of the spectral estimate. The zero frequency intercept is calculated as a function of midband fit and spectral slope, I=β−f_{c}n, where f_{c} is the central frequency of the spectrum. Alternatively, these parameters may also be estimated by using a weighted least-squares method from the spectral estimate. Spectral parameters may be averaged over spatially adjoining samples in depth and/or lateral directions to comprise one image voxel. The spectral parameters are calculated for every voxel in the field of regard of the ultrasound transmission. The time course of the spectral parameters in each voxel will exhibit cyclic oscillations with a period equivalent to that of the cardiac cycle. The time courses for spectral parameters from multiple cardiac cycles may be time synchronized and averaged together. The time synchronization can be achieved through time course resampling or phase adjustments using the peak systolic time instant as a reference. The electrocardiogram signal can be used to obtain a reference indicator of the phases of the cardiac cycle. The duration of the cardiac cycle can also be derived from the intrinsic period of the signals derived from, brain tissue motion. The time course, and intrinsic features thereof, of the backscattered intensity across the cardiac cycle are compared for each voxel against: 1) previously-stored known variations in normal brain tissue, 2) previously stored patterns or time courses of abnormal brain tissue, 3) previously stored historical patterns or time courses from the same subject, if available, for monitoring treatment. Any number of classification algorithms (including but not limited to Bayesian, neural network, support vector machines, k-nearest neighbor, and binary decision) can be used to determine whether the observed brain tissue region exhibits (a) normal pulsation, (b) abnormal pulsation characteristic of brain tissue affected by ischemic stroke, (c) abnormal pulsation characteristic of brain tissue affected by hemorrhagic stroke or (d) unknown abnormal pulsation.

In another aspect, the signal analyzer unit 126 can estimate the statistical properties of the backscattered ultrasound echoes from a region of brain tissue, and calculate a number of parameters describing the statistical properties of the reflected ultrasound signal. The signal analyzer unit 126 conditions the analog backscattered signal for digitization by the ADC 117 through the operations of amplification and of filtering. The envelope of the backscattered radiofrequency signal is determined by one of several methods previously described in the art, including computing the root-mean-squared amplitude, or by converting the signal to analytic form through a Hilbert transform or quadrature filtering operation and computing the magnitude. The histogram of the backscattered intensities from a local region of brain tissue is then computed. The histogram of backscattered intensities may draw from samples taken from multiple cardiac cycles. The samples may be time synchronized and averaged together. The time synchronization can be achieved through time course resampling or phase adjustments using the peak systolic time instant as a reference. The electrocardiogram signal can be used to obtain a reference indicator of the phases of the cardiac cycle. The duration of the cardiac cycle can also be derived from the intrinsic period of the signals derived from brain tissue motion. The similarity of the histogram to a known probability distribution function is then determined using a similarity measure, such as the maximum likelihood function, and maximizing the similarity through an optimization algorithm. Examples of known probability distribution functions include, but are not limited to, the Rayleigh distribution, the Nakagami distribution, the gamma distribution, the Homodyned-K distribution, or a mixture of such distributions. Examples of optimization algorithms that can be used to maximize similarity include, but are not limited to, the quasi-Newton algorithm. The parameters of the probability distribution function that is most similar to the histogram of backscattered intensities are calculated for every voxel in the field of regard of the ultrasound transmission. The time course of the parameters in each voxel will exhibit cyclic oscillations with a period equivalent to that of the cardiac cycle. The time course, and intrinsic features thereof, of the parameters across the cardiac cycle are compared for each voxel against: 1) previously stored known values, and their variations in normal brain tissue, 2) previously stored patterns or time courses of abnormal brain tissue, 3) previously-stored historical values and patterns or time courses from the same subject, if available, for monitoring treatment. Any number of classification algorithms (including but not limited to Bayesian, neural network, support vector machines, k-nearest neighbor, and binary decision) can be used to determine whether the observed brain tissue region exhibits (a) normal pulsation, (b) abnormal pulsation characteristic of brain tissue affected by ischemic stroke, (c) abnormal pulsation characteristic of brain tissue affected by hemorrhagic stroke or (d) unknown abnormal pulsation.

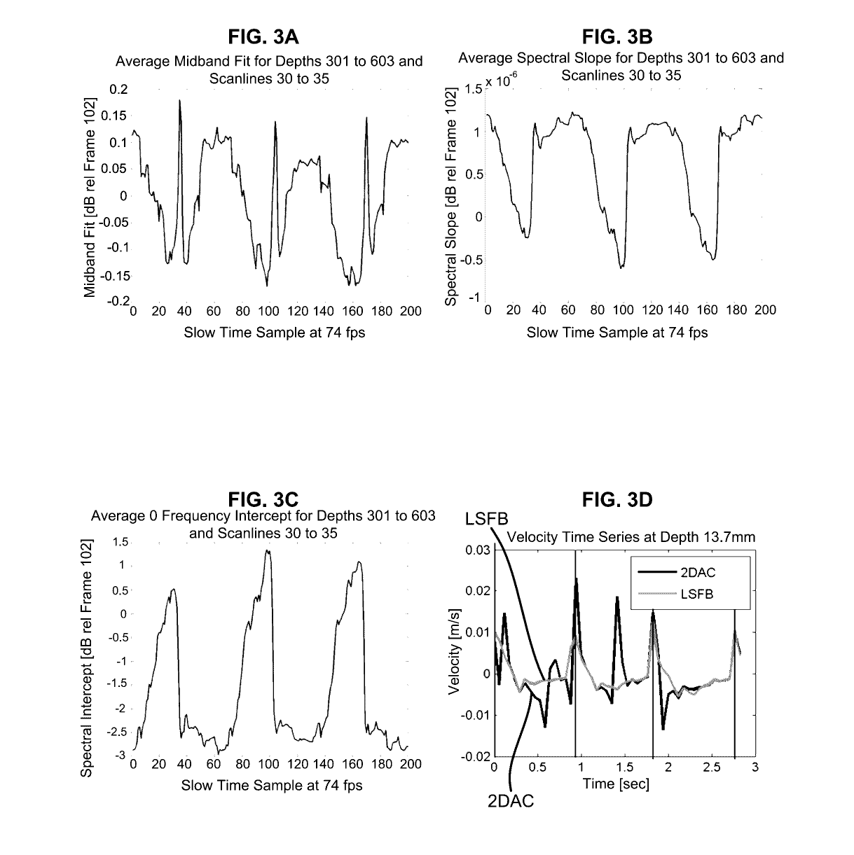

FIG. 3A illustrates an example of the variation of the spectral parameter mid-band-fit during the cardiac cycle in a region of normal brain tissue. FIG. 3B illustrates an example of the variation of the spectral parameter spectral slope during the cardiac cycle in a region of normal brain tissue. FIG. 3C illustrates an example of the variation of the spectral parameter zero-frequency-intercept during the cardiac cycle in a region of normal brain tissue.

Returning to FIG. 1, in another aspect, the signal analyzer unit 126 can filter received ultrasound echoes using a bank of bandpass filters, and estimate a phase shift between consecutive filtered ultrasound echoes to calculate a variation of phase shift with instantaneous frequency contained in the received ultrasound echo. Based on this calculation, the signal analyzer unit 126 can estimate a pulsation of a region of brain tissue that is robust to a number of sources of noise. The signal analyzer unit 126 can further calculate a variation of this pulsation during the cardiac cycle, and compare these parameters and their variation between brain regions, and can compare these parameters against: 1) previously stored known variations in normal brain tissue, 2) previously stored patterns or time courses of abnormal brain tissue, 3) previously stored historical patterns or time courses from a specific subject. A classification algorithm is then used to determine whether the observed brain tissue region exhibits (a) normal pulsation, (b) abnormal pulsation characteristic of brain tissue affected by ischemic stroke, (c) abnormal pulsation characteristic of brain tissue affected by hemorrhagic stroke or (d) unknown abnormal pulsation. FIG. 3D illustrates a time course of estimated brain tissue velocity for three cardiac cycles using the disclosed phase shift method (least squares fit of filter bank outputs, LSFB) compared to method described in the prior art (two-dimensional autocorrelation 2DAC).

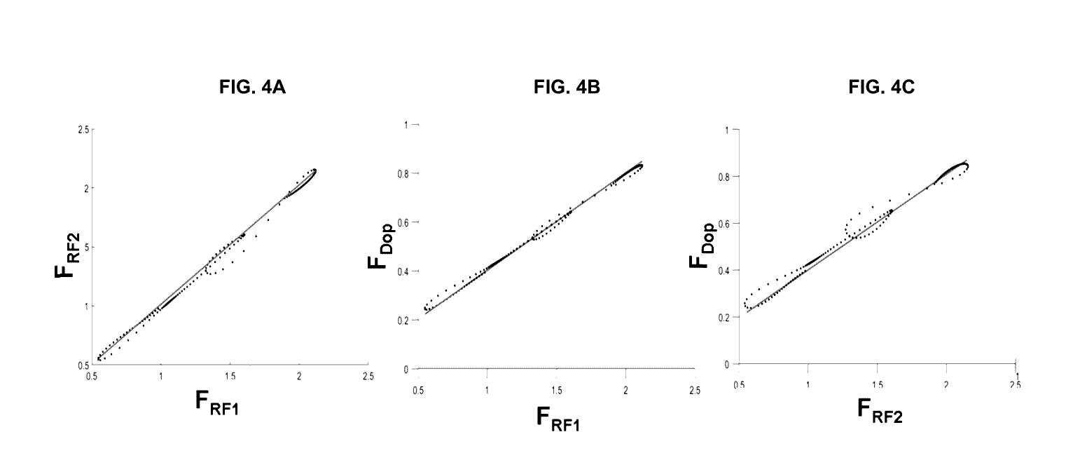

FIG. 4A, FIG. 4B, and FIG. 4C illustrate a 3D geometric view of constrained filtered signals from a pair of consecutive reflected ultrasound echoes. The filter bank output for each filter for a single depth is shown by dots. The solution of the estimator is shown in green as a solid line through the distribution of filter bank outputs. The subsequent component images show: FIG. 4A: RF frequency from pulse 1 to pulse 2, FIG. 4B: RF frequency of pulse 1 to Doppler frequency, and FIG. 4C: RF frequency of pulse 2 to Doppler frequency. The slope of the 3D scatterplot can be used as a parametric measure of the instantaneous velocity of pulsation of the brain at that location.

Returning to FIG. 1, according to an aspect, the image generating unit 121 may generate a three-dimensional (3D) ultrasound image via volume-rendering with respect to volume data and may also generate an elasticity image by imaging deformation of the object 110 due to pressure. Furthermore, the image generating unit 121 may display various pieces of additional information in an ultrasound image by using text and graphics. In addition, the generated ultrasound image may be stored in the memory 106.

The display unit 105 displays the generated ultrasound image. The display unit 105 may display not only an ultrasound image, but also various pieces of information processed by the ultrasound apparatus 100 on a screen image via a graphical user interface (GUI). In addition, the ultrasound apparatus 100 may include two or more displays 105 according to aspects.

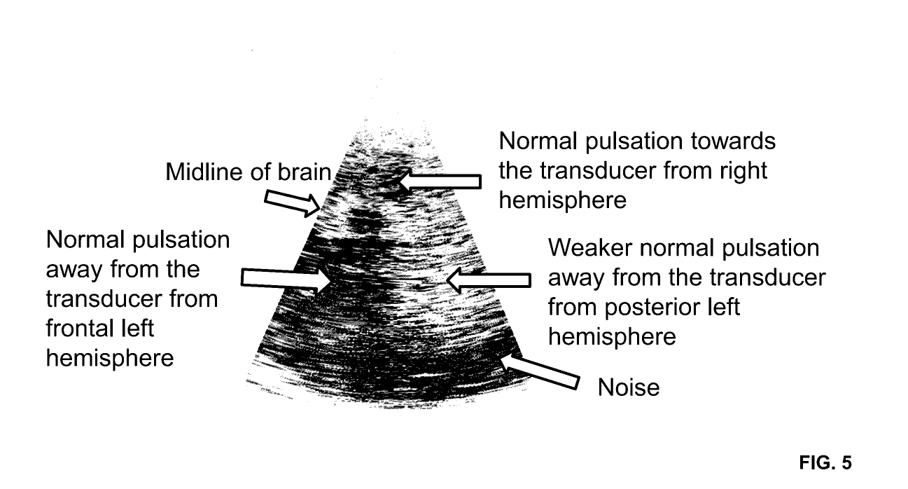

The display unit 105 can also display one or more results of the signal analyzer unit 126. In an aspect, the display unit 105 can display one or more of a composite spatial map of brain tissue pulsatility and/or a parametric spatial map indicating whether different brain regions exhibit pulsations and tissue properties that are (a) normal, (b) characteristic of ischemic stroke, (c) characteristic of hemorrhagic stroke, or (d) indeterminate. FIG. 5 illustrates an example of a spatial map of pulsatility from a normal subject capable of being displayed via the display unit 105.

In an aspect, a physician can use the information displayed on the display unit 105 to determine the type of stroke suffered by a subject. This information aids the physician in determining the proper course of treatment such as, but not limited to, the application of anticoagulating drugs in the case of ischemic stroke. In another aspect, a physician can use the information displayed on the display unit 105 to determine the degree to which a previous diagnosis has changed. Observations from a region previously determined to be characteristic of stroke that now shows improvement may result in the continued course of treatment. Whereas on the other hand observed degradation or lack of response to a treatment may alter the physicians plan of care.

The communication unit 104 can be connected to a network 124 by wire or wirelessly to communicate with an external device or a server. The communication unit 104 may exchange data with a hospital server or another medical apparatus in a hospital, which is connected thereto via a PACS. Furthermore, the communication unit 104 may perform data communication according to the digital imaging and communications in medicine (DICOM) standard.

The communication unit 104 may transmit or receive data related to diagnosis of an object e.g., an ultrasound image, ultrasound data, and Doppler data of the object, via the network 124 and may also transmit or receive medical images captured by another medical apparatus, e.g., a computed tomography (CT) apparatus, a magnetic resonance imaging (MRI) apparatus, or an X-ray apparatus. Furthermore, the communication unit 104 may receive information about a diagnosis history or medical treatment schedule of a patient from a server and utilizes the received information to diagnose the patient. Furthermore, the communication unit 104 may perform data communication not only with a server or a medical apparatus in a hospital, but also with a portable terminal of a medical doctor or patient.

The communication unit 104 can be connected to the network 124 by wire or wirelessly to exchange data with a server 125 (e.g., a medical apparatus, portable terminal, and the like). The communication unit 104 may include one or more components for communication with external devices. For example, the communication unit 104 may include a local area communication unit, a wired communication unit, and/or a wireless communication unit. The local area communication unit can be a module for local area communication within a predetermined distance. Examples of local area communication techniques according to an aspect may include, but are not limited to, wireless LAN, Wi-Fi, Bluetooth, ZigBee, Wi-Fi Direct (WFD), ultra wideband (UWB), infrared data association (IrDA), Bluetooth low energy (BLE), and near field communication (NFC). The wired communication unit can be a module for communication using electric signals or optical signals. Examples of wired communication techniques according to an aspect may include communication via a twisted pair cable, a coaxial cable, an optical fiber cable, and an Ethernet cable. The wireless communication unit can transmit or receive wireless signals to or from at least one selected from a base station, an external terminal, and a server on a mobile communication network. The wireless signals may be voice call signals, video call signals, or various types of data for transmission and reception of text/multimedia messages.

The memory 106 can store various data processed by the ultrasound apparatus 100. For example, the memory 106 may store medical data related to diagnosis of an object, such as ultrasound data and an ultrasound image that are input or output, and may also store algorithms or programs which are to be executed in the ultrasound apparatus 100. The memory 106 may be any of various storage media, e.g., a flash memory, a hard disk drive, EEPROM, etc. Furthermore, the ultrasound apparatus 100 may utilize web storage or a cloud server that performs the storage function of the memory 106 online.

The input device 107 can be configured to receive one or more user inputs for controlling the ultrasound apparatus 100. The input device 107 may include hardware components, such as a keypad, a mouse, a touch panel, a touch screen, and a jog switch. However, aspects are not limited thereto, and the input device 107 may further include any of various other input units including an electrocardiogram (ECG) measuring module, a respiration measuring module, a voice recognition sensor, a gesture recognition sensor, a fingerprint recognition sensor, an iris recognition sensor, a depth sensor, a distance sensor, etc.

The controller 108 may control one or more operations of the ultrasound apparatus 100. In other words, the controller 108 may control operations among the probe 101, the ultrasound transmission/reception unit 102, the image processing unit 103, the communication unit 104, the memory 106, and the input device 107 shown in FIG. 1.

All or some of the probe 101, the ultrasound transmission/reception unit 102, the image processing unit 103, the communication unit 104, the memory 106, the input device 107, and the controller 108 may be implemented as software modules. However, aspects are not limited thereto, and some of the components stated above may be implemented as hardware modules. Furthermore, at least one selected from the ultrasound transmission/reception unit 102, the image processing unit 103, and the communication unit 104 may be included in the controller 108. However, embodiments of the present methods, systems, and apparatuses are not limited thereto.

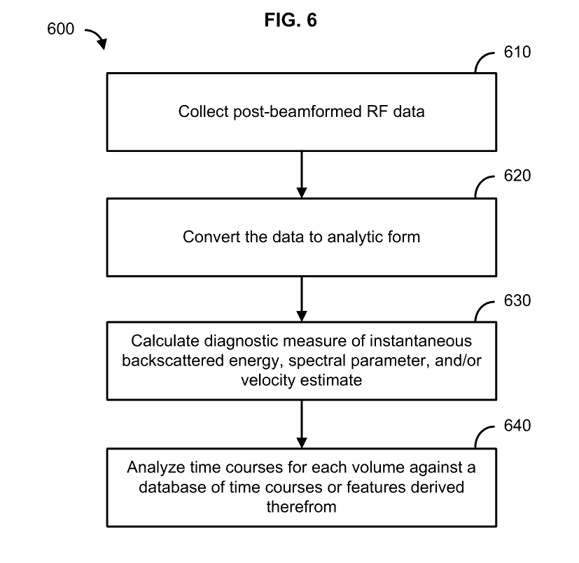

In an aspect, illustrated in FIG. 6, provided is a method 600 that represents at least a portion of the collection and analysis steps described with relation the ultrasound apparatus 100 in FIG. 1. In block 610, post-beamformed RF data is collected. In block 620, the diagnostic measure of instantaneous backscattered energy, spectral parameter, and/or velocity estimate is calculated for each volume under analysis. In block 630, the time courses for each volume are analyzed against the database of time courses or features derived therefrom.

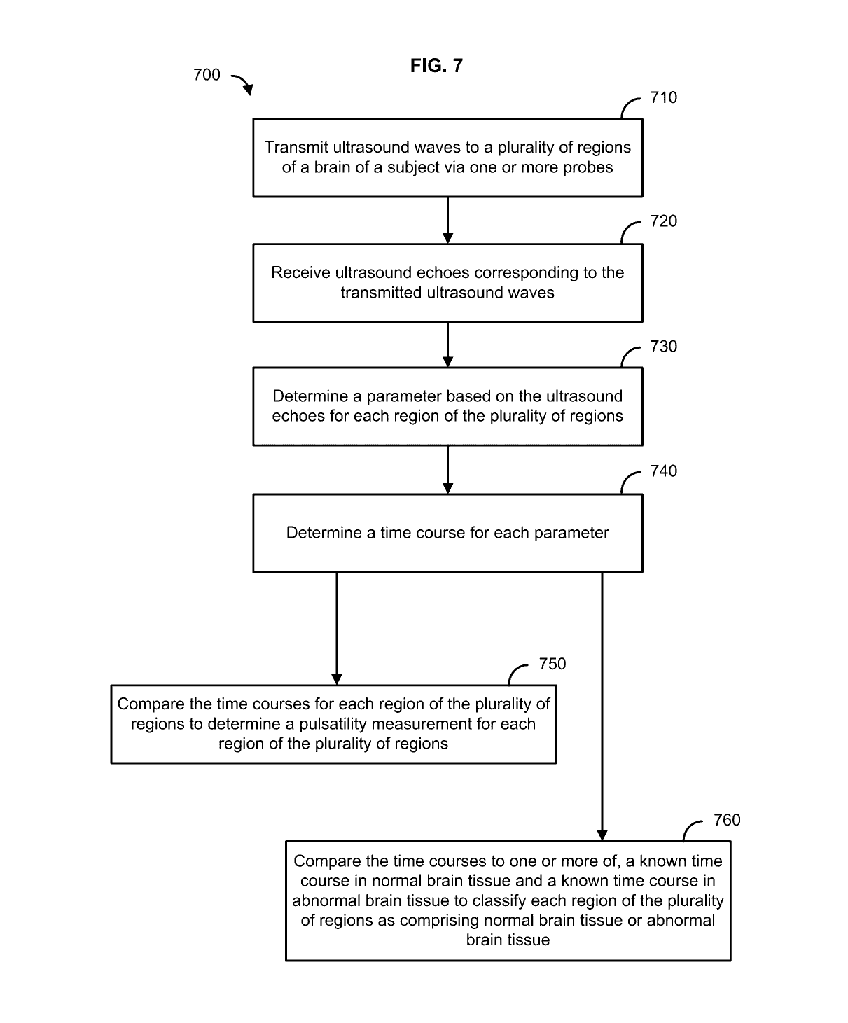

In an aspect, illustrated in FIG. 7, provided is a method 700 comprising transmitting ultrasound waves to a plurality of regions of a brain of a subject via one or more probes at block 710. The method 700 can comprise receiving ultrasound echoes corresponding to the transmitted ultrasound waves at block 720. The method 700 can comprise determining a parameter based on the ultrasound echoes for each region of the plurality of regions at block 730. The parameter can comprise one or more of, a backscattered intensity, a measure derived from the probability distribution of backscattered intensities from a local brain region, a spectral slope of an instantaneous frequency of each ultrasound echo, a mid-band fit of an instantaneous frequency of each ultrasound echo, a zero-frequency offset of an instantaneous frequency of each ultrasound echo, and a phase shift across different frequencies. The method 700 can comprise determining a time course for each parameter at block 740.

In an aspect, the method 700 can proceed to one or both of block 750 and block 760. At block 750, the method 700 can comprise comparing the time courses for each region of the plurality of regions to determine a pulsatility measurement for each region of the plurality of regions. At block 760, the method 700 can comprise comparing the time courses to one or more of, a known time course in normal brain tissue and a known time course in abnormal brain tissue to classify each region of the plurality of regions as comprising normal brain tissue or abnormal brain tissue. The known time course in abnormal brain tissue can comprise a known time course associated with brain tissue affected by ischemic stroke and a known time course associated with brain tissue affected by hemorrhagic stroke.

The method 700 can further comprise receiving a signal from an electrocardiogram to determine the timing of a cardiac cycle, and a timing of brain tissue pulsations relative to the cardiac cycle, and differentiating between normal and abnormal brain tissue by comparing pulsations during a certain portion of the cardiac cycle, and/or the delay between the peak of the pulsations to the beginning of the cardiac cycle.

The method 700 can further comprise filtering the backscattered ultrasound echoes through one or more bandpass filters to determine the phase shift across different frequencies.

The method 700 can further comprise accessing a database comprising a plurality of known time courses in the subject and determining a measure of degree to which the time course has changed over time relative to the plurality of known time courses.

The method 700 can further comprise outputting a composite spatial map of brain tissue pulsatility based on the pulsatility measurements. The method 700 can further comprise outputting a parametric spatial map indicating whether each region of the plurality of regions is one of, normal, characteristic of ischemic stroke, characteristic of hemorrhagic stroke, or indeterminate.

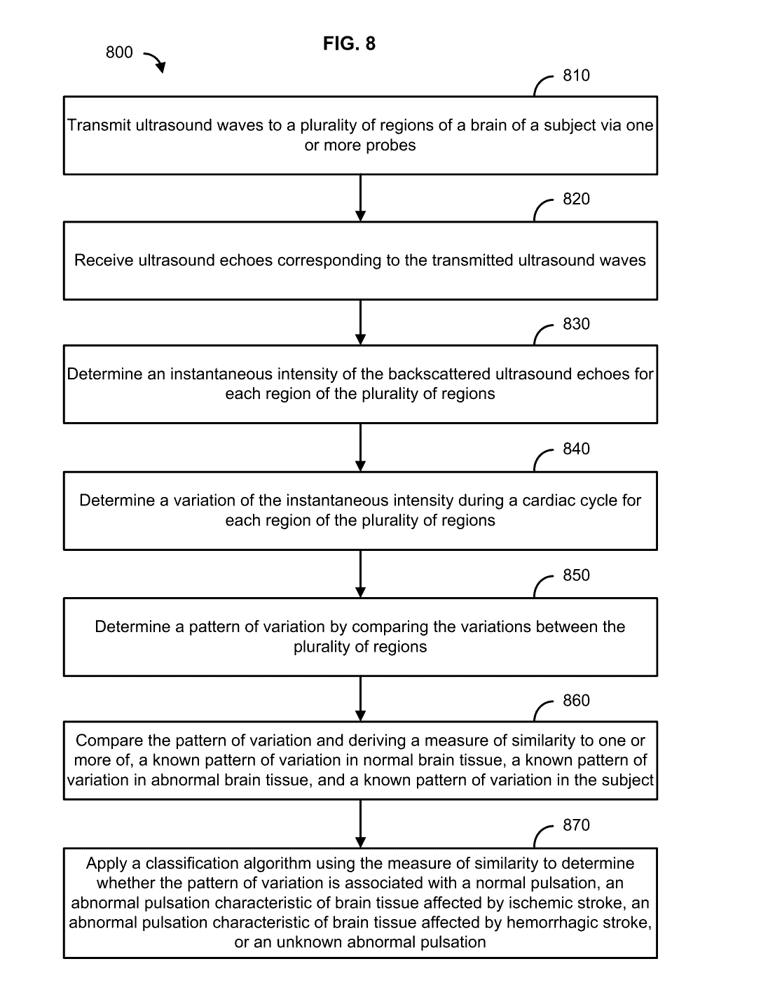

In another aspect, illustrated in FIG. 8, provided is a method 800 comprising transmitting ultrasound waves to a plurality of regions of a brain of a subject via one or more probes at block 810. The method 800 can comprise receiving backscattered ultrasound echoes corresponding to the transmitted ultrasound waves at block 820. The method 800 can comprise determining an instantaneous intensity of the backscattered ultrasound echoes for each region of the plurality of regions at block 830. The method 800 can comprise determining a variation of the instantaneous intensity during a cardiac cycle for each region of the plurality of regions at block 840. The method 800 can comprise determining a pattern of variation by comparing the variations between the plurality of regions at block 850. The method 800 can comprise comparing the pattern of variation and deriving a measure of similarity to one or more of, a known pattern of variation in normal brain tissue, a known pattern of variation in abnormal brain tissue, and a known pattern of variation in the subject at block 860. The method 800 can comprise applying a classification algorithm using the measure of similarity to determine whether the pattern of variation is associated with a normal pulsation, an abnormal pulsation characteristic of brain tissue affected by ischemic stroke, an abnormal pulsation characteristic of brain tissue affected by hemorrhagic stroke, or an unknown abnormal pulsation at block 870.

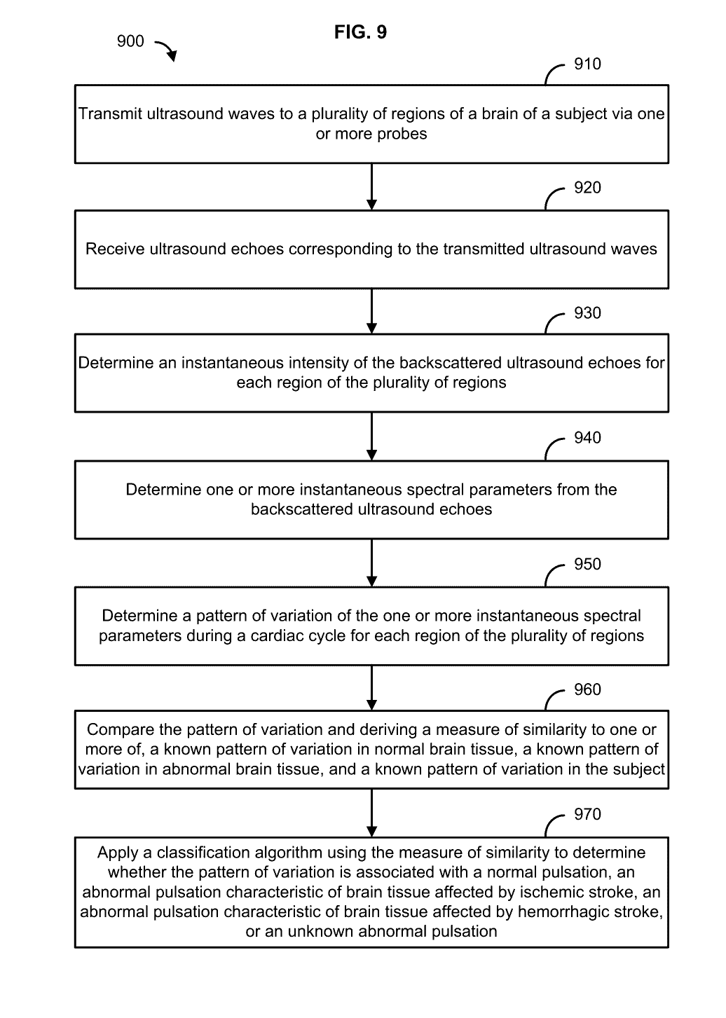

In another aspect, illustrated in FIG. 9, provided is a method 900 comprising transmitting ultrasound waves to a plurality of regions of a brain of a subject via one or more probes at block 910. The method 900 can comprise receiving backscattered ultrasound echoes corresponding to the transmitted ultrasound waves at block 920. The method 900 can comprise determining one or more instantaneous spectral parameters from the backscattered ultrasound echoes at block 930. The method 900 can comprise determining a pattern of variation of the one or more instantaneous spectral parameters during a cardiac cycle for each region of the plurality of regions at block 940. The method 900 can comprise comparing the pattern of variation and deriving a measure of similarity to one or more of, a known pattern of variation in normal brain tissue, a known pattern of variation in abnormal brain tissue, and a known pattern of variation in the subject at block 950. The method 900 can comprise applying a classification algorithm using the measure of similarity to determine whether the pattern of variation is associated with a normal pulsation, an abnormal pulsation characteristic of brain tissue affected by ischemic stroke, an abnormal pulsation characteristic of brain tissue affected by hemorrhagic stroke, or an unknown abnormal pulsation at block 960.

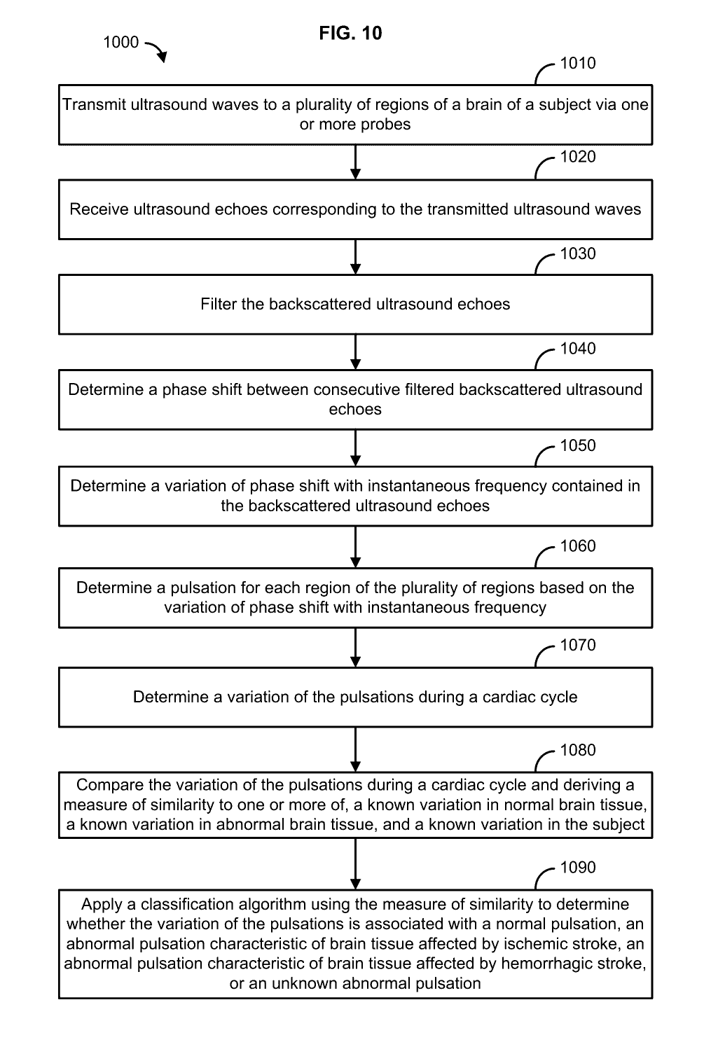

In another aspect, illustrated in FIG. 10, provided is a method 1000 comprising transmitting ultrasound waves to a plurality of regions of a brain of a subject via one or more probes at block 1010.

The method 1000 can comprise receiving backscattered ultrasound echoes corresponding to the transmitted ultrasound waves at block 1020.

The method 1000 can comprise filtering the backscattered ultrasound echoes at block 1030. The method 1000 can comprise determining a phase shift between consecutive filtered backscattered ultrasound echoes at block 1040.

The method 1000 can comprise determining a variation of phase shift with instantaneous frequency contained in the backscattered ultrasound echoes at block 1050. The method 1000 can comprise determining a pulsation for each region of the plurality of regions based on the variation of phase shift with instantaneous frequency at block 1060. The method 1000 can comprise determining a variation of the pulsations during a cardiac cycle at block 1070.

The method 1000 can comprise comparing the variation of the pulsations during a cardiac cycle and deriving a measure of similarity to one or more of, a known variation in normal brain tissue, a known variation in abnormal brain tissue, and a known variation in the subject at block 1080.

The method 1000 can comprise applying a classification algorithm using the measure of similarity to determine whether the variation of the pulsations is associated with a normal pulsation, an abnormal pulsation characteristic of brain tissue affected by ischemic stroke, an abnormal pulsation characteristic of brain tissue affected by hemorrhagic stroke, or an unknown abnormal pulsation at block 1090.

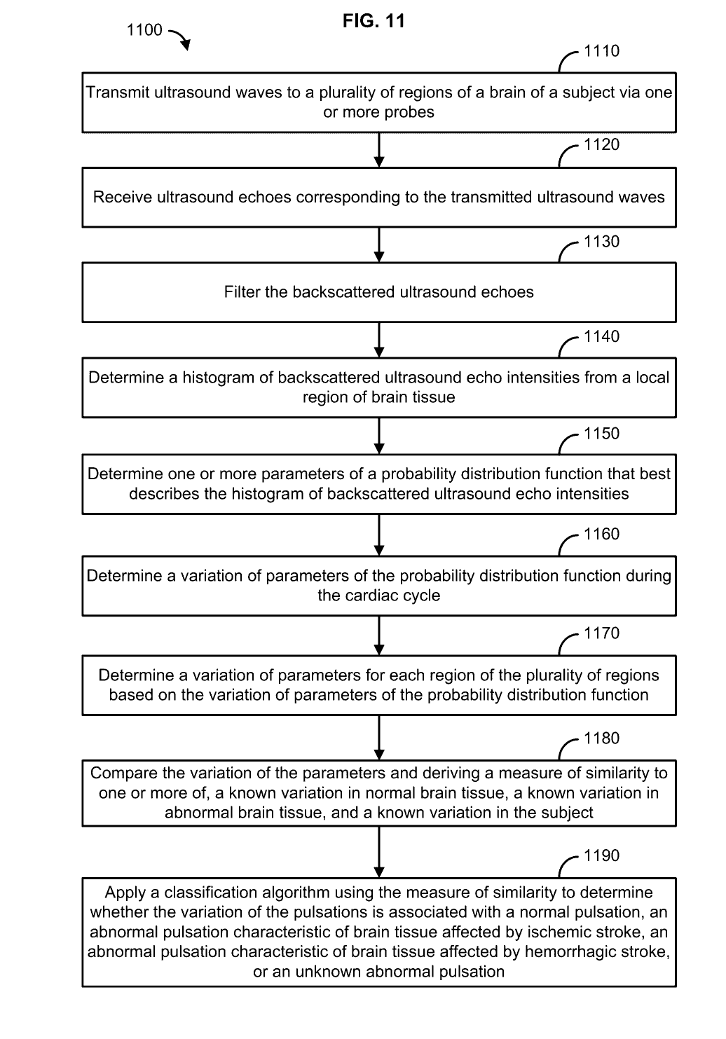

In another aspect, illustrated in FIG. 11, provided is a method 1100 comprising transmitting ultrasound waves to a plurality of regions of a brain of a subject via one or more probes at block 1110. The method 1100 can comprise receiving backscattered ultrasound echoes corresponding to the transmitted ultrasound waves at block 1120. The method 1100 can comprise determining a histogram of backscattered ultrasound echo intensities from a local region of brain tissue at block 1130. The method 1100 can comprise determining one or more parameters of a probability distribution function that best describes the histogram of backscattered ultrasound echo intensities at block 1140. The method 1100 can comprise determining a variation of parameters of the probability distribution function during the cardiac cycle at block 1150. The method 1100 can comprise determining a variation of parameters for each region of the plurality of regions based on the variation of parameters of the probability distribution function at block 1160. The method 1100 can comprise comparing the variation of the parameters and deriving a measure of similarity to one or more of, a known variation in normal brain tissue, a known variation in abnormal brain tissue, and a known variation in the subject at block 1170. The method 1100 can comprise applying a classification algorithm using the measure of similarity to determine whether the variation of the pulsations is associated with a normal pulsation, an abnormal pulsation characteristic of brain tissue affected by ischemic stroke, an abnormal pulsation characteristic of brain tissue affected by hemorrhagic stroke, or an unknown abnormal pulsation at block 1180.

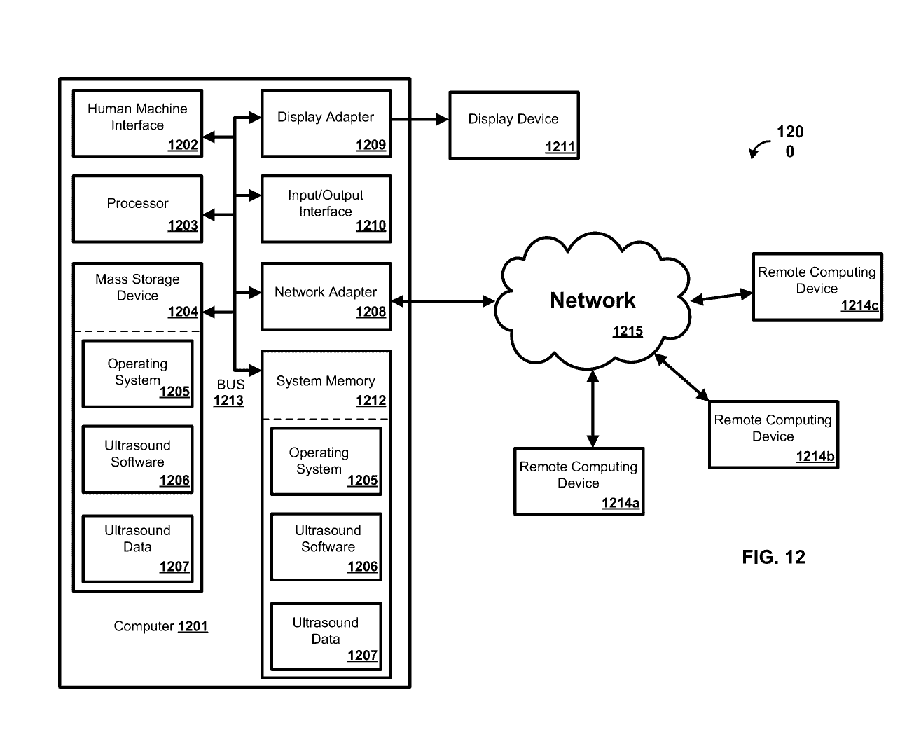

In an exemplary aspect, the methods and systems can be implemented on a computer 1201 as illustrated in FIG. 12 and described below. By way of example, the ultrasound apparatus 100 and/or the server 125 of FIG. 1 can be a computer 1201 as illustrated in FIG. 6. Similarly, the methods and systems disclosed can utilize one or more computers to perform one or more functions in one or more locations. FIG. 12 is a block diagram illustrating an exemplary operating environment 1200 for performing the disclosed methods. This exemplary operating environment 1200 is only an example of an operating environment and is not intended to suggest any limitation as to the scope of use or functionality of operating environment architecture. Neither should the operating environment 1200 be interpreted as having any dependency or requirement relating to any one or combination of components illustrated in the exemplary operating environment 1200.

The present methods and systems can be operational with numerous other general purpose or special purpose computing system environments or configurations. Examples of well known computing systems, environments, and/or configurations that can be suitable for use with the systems and methods comprise, but are not limited to, personal computers, server computers, laptop devices, and multiprocessor systems. Additional examples comprise set top boxes, programmable consumer electronics, network PCs, minicomputers, mainframe computers, distributed computing environments that comprise any of the above systems or devices, and the like.

The processing of the disclosed methods and systems can be performed by-software components. The disclosed systems and methods can be described in the general context of computer-executable instructions, such as program modules, being executed by one or more computers or other devices. Generally, program modules comprise computer code, routines, programs, objects, components, data structures, and/or the like that perform particular tasks or implement particular abstract data types. The disclosed methods can also be practiced in grid-based and distributed computing environments where tasks are performed by remote processing devices that are linked through a communications network. In a distributed computing environment, program modules can be located in local and/or remote computer storage media including memory storage devices.

Further, one skilled in the art will appreciate that the systems and methods disclosed herein can be implemented via a general-purpose computing device in the form of a computer 1201. The computer 1201 can comprise one or more components, such as one or more processors 1203, a system memory 1212, and a bus 1213 that couples various components of the computer 1201 including the one or more processors 1203 to the system memory 1212. In the case of multiple processors 1203, the system can utilize parallel computing.

The bus 1213 can comprise one or more of several possible types of bus structures, such as a memory bus, memory controller, a peripheral bus, an accelerated graphics port, and a processor or local bus using any of a variety of bus architectures. By way of example, such architectures can comprise an Industry-Standard Architecture (ISA) bus, a Micro Channel Architecture (MCA) bus, an Enhanced ISA (EISA) bus, a Video Electronics Standards Association (VESA) local bus, an Accelerated Graphics Port (AGP) bus, and a Peripheral Component Interconnects (PCI), a PCT-Express bus, a Personal Computer Memory Card Industry Association (PCMCIA), Universal Serial Bus (USB) and the like. The bus 1213, and all buses specified in this description can also be implemented over a wired or wireless network connection and one or more of the components of the computer 1201, such as the one or more processors 1203, a mass storage device 1204, an operating system 1205, ultrasound software 1206, ultrasound data 1207, a network adapter 1208, system memory 1212, an Input/Output Interface 1210, a display adapter 1209, a display device 1211, and a human machine interface 1202, can be contained within one or more remote computing devices 1214a,b,c at physically separate locations, connected through buses of this form, in effect implementing a fully distributed system.

The computer 1201 typically comprises a variety of computer readable media. Exemplary readable media can be any available media that is accessible by the computer 1201 and comprises, for example and not meant to be limiting, both volatile and non-volatile media, removable and non-removable media. The system memory 1212 can comprise computer readable media in the form of volatile memory, such as random access memory (RAM), and/or non-volatile memory, such as read only memory (ROM). The system memory 1212 typically can comprise data such as ultrasound data 1207 and/or program modules such as operating system 1205 and ultrasound software 1206 that are accessible to and/or are operated on by the one or more processors 1203.

In another aspect, the computer 1201 can also comprise other removable/non-removable, volatile/non-volatile computer storage media. The mass storage device 1204 can provide non-volatile storage of computer code, computer readable instructions, data structures, program modules, and other data for the computer 1201. For example, a mass storage device 1204 can be a hard disk, a removable magnetic disk, a removable optical disk, magnetic cassettes or other magnetic storage devices, flash memory cards, CD-ROM, digital versatile disks (DVD) or other optical storage, random access memories (RAM), read only memories (ROM), electrically erasable programmable read-only memory (EEPROM), and the like.

Optionally, any number of program modules can be stored on the mass storage device 1204, including by way of example, an operating system 1205 and ultrasound software 1206. One or more of the operating system 1205 and ultrasound software 1206 (or some combination thereof) can comprise elements of the programming and the ultrasound software 1206. Ultrasound data 1207 can also be stored on the mass storage device 1204. Parameters derived from the ultrasound data 1207 can be stored in any of one or more databases known in the art. Examples of such databases comprise, DB2®, Microsoft® Access, Microsoft® SQL Server, Oracle®, mySQL, PostgreSQL, SQLite, and the like. The databases can be centralized or distributed across multiple locations within the network or local to the device itself. 1215.

In another aspect, the user can enter commands and information into the computer 1201 via an input device (not shown). Examples of such input devices comprise, but are not limited to, a keyboard, pointing device (e.g., a computer mouse, remote control), a microphone, a joystick, a scanner, tactile input devices such as gloves, and other body coverings, motion sensor, and the like. These and other input devices can be connected to the one or more processors 1203 via a human machine interface 1202 that is coupled to the bus 1213, but can be connected by other interface and bus structures, such as a parallel port, game port, an IEEE 1394 Port (also known as a Firewire port), a serial port, network adapter 1208, and/or a universal serial bus (USB).

In yet another aspect, a display device 1211 can also be connected to the bus 1213 via an interface, such as a display adapter 1209. It is contemplated that the computer 1201 can have more than one display adapter 1209 and the computer 1201 can have more than one display device 1211. For example, a display device 1211 can be a monitor, an LCD (Liquid Crystal Display), light emitting diode (LED) display, television, smart lens, smart glass, and/or a projector. In addition to the display device 1211, other output peripheral devices can comprise components such as speakers (not shown) and a printer (not shown), which can be connected to the computer 1201 via Input/Output Interface 1210. Any step and/or result of the methods can be output in any form to an output device. Such output can be any form of visual representation, including, but not limited to, textual, graphical, animation, audio, tactile, and the like. The display 1211 and computer 1201 can be part of one device, or separate devices.

The computer 1201 can operate in a networked environment using logical connections to one or more remote computing devices 1214a,b,c. By way of example, a remote computing device 1214a,b,c can be a personal computer, computing station (e.g., workstation), portable computer (e.g., laptop, mobile phone, tablet device), smart device (e.g., smartphone, smart watch, activity tracker, smart apparel, smart accessory), security and/or monitoring device, a server, a router, a network computer, a peer device, edge device or other common network node, and so on. Logical connections between the computer 1201 and a remote computing device 1214a,b,c can be made via a network 1215, such as a local area network (LAN) and/or a general wide area network (WAN). Such network connections can be through a network adapter 1208. A network adapter 1208 can be implemented in both wired and wireless environments. Such networking environments are conventional and commonplace in dwellings, offices, enterprise-wide computer networks, intranets, and the Internet.

For purposes of illustration, application programs and other executable program components such as the operating system 1205 are illustrated herein as discrete blocks, although it is recognized that such programs and components can reside at various times in different storage components of the computing device 1201, and are executed by the one or more processors 1203 of the computer 1201. An implementation of ultrasound software 1206 can be stored on or transmitted across some form of computer readable media. Any of the disclosed methods can be performed by computer readable instructions embodied on computer readable media. Computer readable media can be any available media that can be accessed by a computer. By way of example and not meant to be limiting, computer readable media can comprise “computer storage media” and “communications media.” “Computer storage media” can comprise volatile and non-volatile, removable and non-removable media implemented in any methods or technology for storage of information such as computer readable instructions, data structures, program modules, or other data. Exemplary computer storage media can comprise RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by a computer.

The methods and systems can employ artificial intelligence (AI) techniques such as machine learning and iterative learning. Examples of such techniques include, but are not limited to, expert systems, case based reasoning, Bayesian networks, behavior based AI, neural networks, fuzzy systems, evolutionary computation (e.g. genetic algorithms), swarm intelligence (e.g. ant algorithms), and hybrid intelligent systems (e.g. Expert inference rules generated through a neural network or production rules from statistical learning).

While the methods and systems have been described in connection with preferred embodiments and specific examples, it is not intended that the scope be limited to the particular embodiments set forth, as the embodiments herein are intended in all respects to be illustrative rather than restrictive.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification.

It will be apparent to those skilled in the art that various modifications and variations can be made without departing from the scope or spirit. Other embodiments will be apparent to those skilled in the art from consideration of the specification and practice disclosed herein. It is intended that the specification and examples be considered as exemplary only, with a true scope and spirit being indicated by the following claims.

What is claimed is:

1. A method comprising:
transmitting ultrasound waves to a plurality of regions of a brain of a subject via one or more probes;
receiving ultrasound echoes corresponding to the transmitted ultrasound waves;
determining a parameter based on the ultrasound echoes for each region of the plurality of regions;
determining a time course for each parameter; and
comparing the time courses for each region of the plurality of regions to determine a pulsatility measurement for each region of the plurality of regions.

2. The method of claim 1, further comprising:
comparing the time courses to one or more of, a known time course in normal brain tissue and a known time course in abnormal brain tissue to classify each region of the plurality of regions as comprising normal brain tissue or abnormal brain tissue.

3. The method of claim 1, further comprising receiving a signal from an electrocardiogram to determine the timing of a cardiac cycle, and a timing of brain tissue pulsations relative to the cardiac cycle, and differentiating between normal and abnormal brain tissue by comparing pulsations during a certain portion of the cardiac cycle, and/or the delay between the peak of the pulsations to the beginning of the cardiac cycle.

4. The method of claim 1, wherein the parameter comprises one or more of, a backscattered intensity, a measure derived from the probability distribution of backscattered intensities from a local brain region, a spectral slope of an instantaneous frequency of each ultrasound echo, a mid-band fit of an instantaneous frequency of each ultrasound echo, a zero-frequency offset of an instantaneous frequency of each ultrasound echo, and a phase shift across different frequencies.

5. The method of claim 4, further comprising filtering the backscattered ultrasound echoes through one or more bandpass filters to determine the phase shift across different frequencies.

6. The method of claim 1, wherein the known time course in abnormal brain tissue comprises a known time course associated with brain tissue affected by ischemic stroke and a known time course associated with brain tissue affected by hemorrhagic stroke.

7. The method of claim 1, further comprising:
accessing a database comprising a plurality of known time courses in the subject; and
determining a measure of degree to which the time course has changed over time relative to the plurality of known time courses.

8. The method of claim 1, further comprising outputting a composite spatial map of brain tissue pulsatility based on the pulsatility measurements.

9. The method of claim 1, further comprising outputting a parametric spatial map indicating whether each region of the plurality of regions is one of, normal, characteristic of ischemic stroke, characteristic of hemorrhagic stroke, or indeterminate.

10. A method comprising:
transmitting ultrasound waves to a plurality of regions of a brain of a subject via one or more probes;
receiving ultrasound echoes corresponding to the transmitted ultrasound waves;
determining a parameter based on the ultrasound echoes for each region of the plurality of regions;
determining a time course for each parameter; and
comparing the time courses to one or more of, a known time course in normal brain tissue and a known time course in abnormal brain tissue to classify each region of the plurality of regions as comprising normal brain tissue or abnormal brain tissue.

11. The method of claim 10, further comprising:
comparing the time courses for each region of the plurality of regions to determine a pulsatility measurement for each region of the plurality of regions.

12. The method of claim 10, further comprising receiving a signal from an electrocardiogram to determine the timing of a cardiac cycle, and a timing of brain tissue pulsations relative to the cardiac cycle, and differentiating between normal and abnormal brain tissue by comparing pulsations during a certain portion of the cardiac cycle, and/or the delay between the peak of the pulsations to the beginning of the cardiac cycle.

13. The method of claim 10, wherein the parameter comprises one or more of, a backscattered intensity, a measure derived from the probability distribution of backscattered intensities from a local brain region, a spectral slope of an instantaneous frequency of each ultrasound echo, a mid-band fit of an instantaneous frequency of each ultrasound echo, a zero-frequency offset of an instantaneous frequency of each ultrasound echo, and a phase shift across different frequencies.

14. The method of claim 13, further comprising filtering the backscattered ultrasound echoes through one or more bandpass filters to determine the phase shift across different frequencies.

15. The method of claim 10, wherein the known time course in abnormal brain tissue comprises a known time course associated with brain tissue affected by ischemic stroke and a known time course associated with brain tissue affected by hemorrhagic stroke.

16. The method of claim 10, further comprising:
accessing a database comprising a plurality of known time courses in the subject; and
determining a measure of degree to which the time course has changed over time relative to the plurality of known time courses.

17. The method of claim 10, further comprising outputting a composite spatial map of brain tissue pulsatility based on the pulsatility measurements.

18. The method of claim 10, further comprising outputting a parametric spatial map indicating whether each region of the plurality of regions is one of, normal, characteristic of ischemic stroke, characteristic of hemorrhagic stroke, or indeterminate.

19. A system comprising:
one or more ultrasound transducers configured to transmit ultrasound waves to a plurality of regions of an object and receive backscattered ultrasound echoes corresponding to the transmitted ultrasound waves;
a processor, coupled to the one or more ultrasound transducers, wherein the processor is configured to,
transmit ultrasound waves to a plurality of regions of a brain of a subject via one or more probes;
receive ultrasound echoes corresponding to the transmitted ultrasound waves;
determine a parameter based on the ultrasound echoes for each region of the plurality of regions;
determine a time course for each parameter; and
compare the time courses for each region of the plurality of regions to determine a pulsatility measurement for each region of the plurality of regions.

20. The system of claim 19, wherein the processor is further configured to:
compare the time courses to one or more of, a known time course in normal brain tissue and a known lime course in abnormal brain tissue to classify each region of the plurality of regions as comprising normal brain tissue or abnormal brain tissue.
